(19) United States
(12) Patent Application Publication
(10) Pub. No.: US 2024/0009314 A1
(43) Pub. Date: Jan. 11, 2024

(54) COMPOSITIONS AND METHODS FOR TARGETING TUMOR-ASSOCIATED MACROPHAGES

(71) Applicant: Resolute Science, San Diego, CA (US)

(72) Inventor: Faith Hemenway BARNETT, San Diego, CA (US)

(73) Assignee: Resolute Science, San Diego, CA (US)

(21) Appl. No.: 18/022,095

(22) PCT Filed: Aug. 20, 2021
(86) PCT No.: PCT/US2021/046984

§ 371 Date: Feb. 17, 2023

Related U.S. Application Data
(60) Provisional application No. 63/068,904, filed on Aug. 21, 2020.

(51) Int. Cl. A61K 47/61; A61K 47/54; A61K 49/00; A61K 51/06

(52) U.S. Cl. CPC A61K 47/61; A61K 47/549; A61K 47/542; A61K 49/0054; A61K 51/06

(57) ABSTRACT

The present invention relates to compounds that target monocytes, macrophages and other cells (such as dendritic cells) that express CD-206, particularly those cells at are assembled at a site of disease, using a target moiety coupled to a glucan backbone. The compounds disclosed here preferably comprise a glucan backbone, a targeting moiety, a targeting moiety linker, a payload and optionally a payload linker. The present invention also provides methods of making such compounds and compositions. The present invention also provides diagnostic methods and methods of treatment using compounds comprising a target moiety coupled to a glucan backbone.

### CROSS-REFERENCE TO RELATED APPLICATION

This application claims priority from U.S. provisional application No. 63/068,904, filed Aug. 21, 2020, entitled “COMPOSITIONS AND METHODS FOR TARGETING TUMOR-ASSOCIATED MACROPHAGES,” which is incorporated by reference in its entirety for all purposes.

### BACKGROUND OF THE INVENTION

CD206⁺ cells, particularly macrophages, have been targeted by various molecules in the hopes of delivering diagnostic and therapeutic to sites where such cells assemble. One example of such molecules is found in US 2017/0209584, entitled, “Compositions for Targeting Macrophages and Other CD206 High Expressing Cells and Methods of Treating and Diagnosis.” While the molecules disclosed in this reference and others may target the CD206⁺ cells of interest, the molecules suffer from a number of short comings.

### BRIEF SUMMARY OF THE INVENTION

In one aspect, provided is a composition comprising: a CD206 targeting moiety coupled to a glucan backbone comprising a plurality of backbone monomers via a targeting linker comprising a carbamate group and a chain moiety, wherein the carbamate group is connected to a backbone monomer and the chain moiety connects the carbamate group and the CD206 targeting moiety, and an active component coupled to the glucan backbone.

Provided in some aspects are methods of delivering an agent to a macrophage comprising contacting said macrophage with a compound described herein.

Provided in some aspects are methods of treating cancer in a subject comprising administering to the subject a therapeutically effective amount of a compound described herein, wherein the active component is a therapeutic agent.

### BRIEF DESCRIPTION OF THE DRAWINGS

FIG. 1 is a graphic representation of a candidate CD206⁺ targeting molecule labeled with FITC.

FIG. 2 is a is a graphic representation of a candidate CD206⁺ targeting molecule labeled with MMAE.

FIG. 3 is a graph showing the impact of Target 5 at three different concentrations, 0.5 mg/ml (○), 5 mg/ml (▪), and 50 mg/ml (□), of as compared to Temozolomide (▴) and a saline vehicle control (●).

FIG. 4 is a graphic representation of a candidate CD306³⁰ targeting molecule showing a cyclodextrin backbone and potential payloads.

FIG. 5 is a graphic representation of a candidate CD306³⁰ targeting molecule carrying a metal ion chelator.

FIG. 6 is a graph showing tumor growth in a syngeneic murine model of triple negative breast cancer where mice are treated with either Target 5 at 5 mg/kg (○), Target 5 at 15 mg/kg (▴), or Paclitaxel at 15 mg/kg (x).

FIG. 7 is a graph showing survival of mice treated with Target 5 in a U87 intracranial model of glioblastoma as compared to mice administered saline.

FIG. 8A-8C is a graph showing the effect of various concentrations of Target 5 on tumor volume (FIG. 8A), percent tumor volume change (FIG. 8B), and body weight (FIG. 8C) in a GL261 glioma mouse model.

FIG. 9 is a graph showing the effect of Target 5 on tumor volume in a syngeneic murine colon cancer model.

FIG. 10 is a MRI image showing that Target-7 has greater specificity for tumor with potential for less toxicity.

FIG. 11A is a graph showing the signal intensity ratios of post-contrast tumor to selected tissues. FIG. 11B is a graph showing signal to noise ratio (SNR) comparisons by group.

### DETAILED DESCRIPTION OF THE INVENTION

The present invention relates to compounds that target monocytes, macrophages and other cells (such as dendritic cells) that express CD206, particularly those cells that are assembled at a site of disease, using a target moiety coupled to a glucan backbone. The compounds disclosed here preferably comprise a glucan backbone, a targeting moiety, a targeting moiety linker, a payload and optionally a payload linker. The present invention also provides methods of making such compounds and compositions. The present invention also provides diagnostic methods and methods of treatment using compounds comprising a target moiety coupled to a glucan backbone.

### Chemical Definitions

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (-CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbamate” refers to the group —O—C(═O)—NH—. Unless specified otherwise, it is understood that the nitrogen atom of the carbamate group is unsubstituted (i.e., bears a hydrogen atom).

“Oxo” refers to the moiety =O.

“Optionally substituted” unless otherwise specified means that a group may be unsubstituted or substituted by one or more (e.g., 1, 2, 3, 4, 5, 6, 7, 8, 9, 10, 11, or 12) of the substituents listed for that group in which the sub stituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

### Compounds

The compounds disclosed here preferably comprise various components, including a glucan backbone, a targeting moiety, a targeting moiety linker, a payload and optionally a payload linker. The arrangement of these components provides a compound that preferentially targets CD206⁺ cells and is internalized. The ability to be internalized by CD206⁺ cells allows for the disclosed compounds to deliver payloads to disease sites where such cells assemble. Solid tumor cancers and granulomatous diseases often comprise by CD206⁺ cell assemblies. The present application describes improved compositions and methods for imaging and treating solid tumor cancers or granulomatous diseases by targeting the CD206⁺ cells that assemble at or otherwise are associated with these disease states. In certain embodiments, the disclosed compounds can also function as an intra-operative imaging agent, a MRI imaging agent or radiosensitizer, and to deliver radiopharmaceuticals to primary and metastatic cancer cells in the brain and body.

### Glucan Backbone

The compounds described here comprise a glucan backbone, which is a linear, branched, or circular oligosaccharide or polysaccharide comprising a plurality of glucose monomers linked predominantly by C-1→C-6 glycosidic bonds. Other glycosidic bonds such as α-1,3 or α-1,4 linkages may also be present. A glucan backbone may also be defined as a polymer of glucose wherein the position of glycosidic bonds is varied. A glucan backbone may comprise the alpha or the beta isomer of glucose. Examples of glucan backbones include dextran, a linear or branched compound, and cyclodextrin, a circular glucan.

A glucan backbone may vary in mass and molecular weight, as determined in part by the number of glucose monomers. In some embodiments, a glucan backbone may range in molecular weight from 1-30 kilodaltons (kDa). Preferred embodiments include glucan backbones of approximately 1 kDa, 3 kDa, 6 kDa, 10 kDa, 20 kDa, or 30 kDa. In some embodiments, the glucan backbone may range in molecular mass from 1,000 to 30,000 grams per mole (g/mol). In some embodiments, the glucan backbone may contain glucose monomers ranging from 5 to 167 in number. The glucan backbone can be linear, branched, circular, or combinations thereof. For example, dextran is an example of a linear or branched glucan backbone. Cyclodextrin is another example of a glucan backbone. The backbones described here can be substituted or unsubstituted. For example, a substituted cyclodextrin is a cyclodextrin derivative that is hydrophobic, hydrophilic, ionized, non-ionized, or any other variation thereof.

### Targeting Moiety

The compounds disclose here comprise a targeting moiety coupled to a glucan backbone. In some embodiments, the targeting moiety is a CD206 targeting moiety. In some embodiments of the above aspects, the targeting moiety is a CD206 ligand. A targeting moiety is a molecule, a compound, a structure, or any combination thereof that targets one or more pattern recognition receptors on CD206⁺ cells. The targeting moiety may target a pattern recognition receptor that is also be characterized as a C-type lectin receptor. Preferably, the targeting moiety targets CD206, a mannose receptor. The targeting moiety may target one or more CD206⁺ cells, particularly CD206⁺ monocytes and macrophages. In some embodiments, the targeting moiety is or comprises a CD206 ligand. In some embodiments, the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate. A preferred CD206 ligand is mannose, D- and L-isomers thereof, and furanoses (5-membered rings) and pyranoses (6-membered rings) thereof.

In some embodiments, the targeting moieties are attached to between about 10% and about 50% of the glucose residues of the glucan backbone, or between about 20% and about 45% of the glucose residues, or between about 25% and about 40% of the glucose residues. (It should be noted that the MWs referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.)

### Ratio of Targeting Linker to Backbone

The density of a targeting moiety relative to backbone subunits is presented using a targeting moiety to backbone subunit ratio for linear and branched polysaccharide backbones. Degree of substitution (d.s.) is used to communicate the density of targeting moieties on circular backbones. The ratio of a targeting moiety to a glucan backbone refers to the number of targeting moieties that substitute a backbone subunit or subunits. For example, a ratio of 1:7 or 1 to 7 means that there is one targeting moiety for every seven glucose subunits in a glucan backbone. The d.s. describes the average number of substituents or substituted positions per unit base. For example, a d.s. of 0.9 means that one backbone subunit is substituted with an average of 0.9 targeting moieties. In some embodiments, the targeting moiety to backbone subunit ratio is from about 1:5 to about 1:25. In some embodiments, the targeting moiety to backbone subunit ratio is from about 1:6 to about 1:19. In some embodiments, the d.s. is from about 0.1 to about 7. In some embodiments, the d.s. is from about 0.5 to 5.

### Targeting Linker

A targeting linker is a cleavable or a non-cleavable linker that connects a glucan backbone to a targeting moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The targeting linker may comprise a carbamate group. In some embodiments, the targeting linker comprises a carbamate group and a chain moiety, wherein the carbamate group is connected to a backbone monomer and the chain moiety connects the carbamate group and the targeting moiety. Herein, a carbamate functional group takes the plain and ordinary meaning derived from the field of organic chemistry. In some embodiments, the chain moiety of the targeting linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more CD206 targeting moieties are attached to the glucan backbone through a linker. The linker may be attached at from about 1 to about 50% of the backbone moieties.

### Active Component

An active component is a molecule or a compound that may be used for diagnostic purposes, therapeutic purposes, or a combination thereof. An active component is also referred to as a payload. An active component may be or comprise a cytotoxic agent, an imaging agent, or a combination thereof.

### Diagnostic Payloads

In some embodiments, the active component is an imaging agent. In some embodiments, the imaging agent is 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye6SO, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultrasmall paramagnetic particle, a manganese chelate, gallium containing agent, 64Cu diacetylbis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium sulphate, or a combination thereof. In some embodiments, an imaging agent is conjugated to one or more additional agents, such as a targeting agent, a cytotoxic agent, or a macrophage polarizing agent.

### Therapeutic Payloads

In some embodiments, the active component is a therapeutic agent. The therapeutic agent may be any compound known to be useful for the treatment of a macrophage-mediated disease. Therapeutic agents include, but are not limited to, chemotherapeutic agents, such as doxorubicin; anti-infective agents, such as antibiotics (e.g. tetracycline, streptomycin, and isoniazid), anti-virals, anti-fungals, and anti-parasitics; immunological adjuvants; steroids; nucleotides, such as DNA, RNA, RNAi, siRNA, CpG or Poly (I:C); peptides; proteins; or metals such as silver, gallium or gadolinium.

In certain embodiments, the therapeutic agent is an antimicrobial drug selected from the group comprising or consisting of: an antibiotic; an anti-tuberculosis antibiotic (such as isoniazid, streptamycin, or ethambutol); an anti-viral or anti-retroviral drug, for example an inhibitor of reverse transcription (such as zidovudin) or a protease inhibitor (such as indinavir); drugs with effect on leishmaniasis (such as Meglumine antimoniate). In certain embodiments, the therapeutic agent is an anti-microbial active, such as amoxicillin, ampicillin, tetracyclines, aminoglycosides (e.g., streptomycin), macrolides (e.g., erythromycin and its relatives), chloramphenicol, ivermectin, rifamycins and polypeptide antibiotics (e.g., polymyxin, bacitracin) and zwittermicin. In certain embodiments, the therapeutic agent is selected from isoniazid, doxorubicin, streptomycin, and tetracycline.

In some embodiments, the therapeutic agent comprises a high energy killing isotope which has the ability to kill macrophages and tissue in the surrounding macrophage environment. Suitable radioisotopes include: ^{210/212/213/214}Bi , ^{131/140}Ba, ^{11/14}C, ⁵¹Cr, ^{67/68}Ga, ¹⁵³Gd , ⁹⁹mTc, ^{88/90/91}Y, ^{123/124/125/131}I, ^{111/115} mln, ¹⁸F, ¹⁰⁵Rh, ¹⁵³Sm, ⁶⁷Cu, ¹⁶⁶Ho, ¹⁷⁷Lu, ¹⁸⁶ Re and ¹⁸⁸Re, ^{32/33}P, ^{46/475}Sc, ⁷²¹⁷⁵Se, ³⁵S, ¹⁸²Ta, ^{127/129/132}Te, ⁶⁵ Zn and ^{89/95}Zr.

In other embodiments, the therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr.

In still further embodiments, the therapeutic agent is selected from the group consisting of cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radio sensitizing drugs. By way of more specific example, the therapeutic agent is selected from the group consisting of temozolomide, mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, isoniazid, indomethacin, gallium(III), 68gallium(III), aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, and dynemicins.

In embodiments wherein the therapeutic agent is a hormone or hormone antagonist, the therapeutic agent may be selected from the group consisting of prednisone, hydroxyprogesterone, medroprogesterone, diethylstilbestrol, tamoxifen, testosterone, and aminogluthetimide.

In embodiments wherein the therapeutic agent is a prodrug, the therapeutic agent may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug.

In some embodiments the active component is a cytotoxic agent or comprises a cytotoxic agent. In some embodiments, the cytotoxic agent is a chemotherapeutic agent, an antitubulin agent, a DNA modifying agent, or a small interfering ribonucleic acid. In some embodiments, the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines.

### Payload Linker

In certain embodiments the active component or payload is coupled directly to the glucan backbone. In some embodiments, the active component is connected to a glucan backbone via a linker. The linker can be cleavable or non-cleavable. In some embodiments, the one or more therapeutic agent is attached via a biodegradable linker. In some embodiments, the biodegradable linker is acid sensitive, such as a hydrazone linker. The use of an acid sensitive linker enables the drug to be transported into the cell and allows for the release of the drug substantially inside of the cell. In some embodiments, the payload linker is a Val-Cit linker.

The payload linker may comprise a carbamate group. In some embodiments, the payload linker comprises a carbamate group and a chain moiety, wherein the carbamate group is connected to a backbone monomer and the chain moiety connects the carbamate group and the active component. Herein, a carbamate functional group takes the plain and ordinary meaning derived from the field of organic chemistry. In some embodiments, the chain moiety of the payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O (C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

Secondary payloads and linkers

In addition to the targeting, diagnostic, and therapeutic payloads, the compounds disclosed here can encompass the inclusion of secondary agents that can be coupled to the glucan backbone to add additional functional capabilities. Typically, the secondary payload is coupled to the linker in a manner similar to that used to couple the targeting moiety to the targeting linker.

A secondary payload can encompass, for example, additional agents for imaging, therapy, or for other purposes. Specifically, in one embodiment, combinations of therapeutic and imaging agents can be linked to the glucan backbone to combine diagnostic and therapeutic functionalities. In another embodiment, various amino acids, such as cysteine or lysine can be coupled to the linker to crosslink the molecule to a target.

A secondary payload linker is a cleavable or a non-cleavable linker that connects a glucan backbone to a secondary payload moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The secondary payload linker may comprise a carbamate group. In some embodiments, the secondary payload linker comprises a carbamate group and a chain moiety, wherein the carbamate group is connected to a backbone monomer and the chain moiety connects the carbamate group and the secondary agent. Herein, a carbamate functional group takes the plain and ordinary meaning derived from the field of organic chemistry. In some embodiments, the chain moiety of the secondary payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an 0 atom, a S atom, and an optionally substituted N atom. In some embodiments, the chain moiety comprises a C₁-C₁₂ alkylene chain. In some embodiments, the chain moiety comprises a C₃-C7 alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂haloalkyl, O(C₁-C₁₂ alkyl), O(C₁C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more secondary payload moieties are attached to the glucan backbone through a linker. The linker may be attached at from about 1 to about 50% of the backbone moieties.

### Diagnostic Methods

Diagnostic methods are disclosed for in vivo detection of diseases or conditions using the disclosed compounds. In certain embodiments, the disclosed compounds include a detection. As used herein, the term “detectable label or moiety” means an atom, isotope, or chemical structure which is: (1) capable of attachment to the carrier molecule; (2) non-toxic to humans or other mammalian subjects; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means.

Detection labels include, but are not limited to, fluorescent molecules (a.k.a. fluorochromes and fluorophores), chemiluminescent reagents (e.g., luminol), bioluminescent reagents (e.g., luciferin and green fluorescent protein (GFP)), metals (e.g., gold nanoparticles), and radioactive isotopes (radioisotopes). Suitable detection labels can be selected based on the choice of imaging method. For example, the detection label can be a near infrared fluorescent dye for optical imaging, a gadolinium chelate for MRI imaging, a radionuclide for PET or SPECT imaging, or a gold nanoparticle for CT imaging.

The disclosed compounds can include a detectable label useful for optical imaging. A number of approaches can be used for optical imaging. The various methods depend upon fluorescence, bioluminescence, absorption or reflectance as the source of contrast. Fluorophores are compounds or moieties that absorb energy of a specific wavelength and re-emit energy at a different (but equally specific) wavelength. In certain embodiments, the detectable label is a near-infrared (NIR) fluorophore. Suitable NIRs include, but are not limited to, VivoTag-S.RTM. 680 and 750, Kodak X-SIGHT Dyes and Conjugates, DyLight 750 and 800 Fluors, Cy 5.5 and 7 Fluors, Alexa Fluor 680 and 750 Dyes, and IRDye 680 and 800CW Fluors. In certain embodiments, Quantum dots, with their photostability and bright emissions, can also be used with optical imaging. In certain embodiments, pre-existing surgical microscopes can be adapted for use in “green” channel by adding a filter to the light source.

The disclosed compounds can include a detectable label (e.g., a radionuclide) useful for nuclear medicine imaging. Nuclear medicine imaging involves the use and detection of radioisotopes in the body. Nuclear medicine imaging techniques include scintigraphy, single photon emission computed tomography (SPECT), and positron emission tomography (PET). In these techniques, radiation from the radioisotopes can be captured by a gamma camera to form two-dimensional images (scintigraphy) or 3-dimensional images (SPECT and PET).

The disclosed compounds can be used in combination with molecular imaging to detect cancer cells, such as those that have metastasized and therefore spread to another organ or tissue of the body, using an in vivo imaging device. A non-invasive method is therefore provided for detecting cancer cells in a subject that involves administering a pharmaceutical composition containing the disclosed compounds to the subject and then detecting the biodistribution of disclosed compounds using an imaging device. In some embodiments, the pharmaceutical composition is injected into the parenchyma. In other embodiments, the pharmaceutical composition is injected into the circulation.

The disclosed compounds can also be used for intraoperative detection of cancer. For example, the disclosed compounds can be used for intraoperative lymphatic mapping (ILM) to trace the lymphatic drainage patterns in a cancer patient to evaluate potential tumor drainage and cancer spread in lymphatic tissue. In these embodiments, the disclosed compounds are injected into the tumor and their movement through the lymphatic system is traced using a molecular imaging device. As another example, the disclosed compounds can be used for intraoperative assessment of, for example, tumor margins and tumor adjacent tissues for the presence of cancer cells. This can be useful, for example, in effectively resecting tumors and detecting the spread of cancer proximal to the tumor. In some embodiments, the disclosed compounds are able to crosses the blood-tumor barrier. In some embodiments, the disclosed compounds are able to carry payloads into brain tumors and across the blood-tumor barrier without leaking across the blood-brain barrier.

The disclosed methods of imaging to detect cancer cells are referred to herein as non-invasive. By non-invasive is meant that the disclosed compounds can be detected from outside of the subject's body. By this it is generally meant that the signal detection device is located outside of the subject's body. It is understood, however, that the disclosed compounds can also be detected from inside the subject's body or from inside the subject's gastrointestinal tract or from inside the subject's respiratory system and that such methods of imaging are also specifically contemplated. For example, for intraoperative detection, the signal detection device can be located either outside or inside of the subject's body. From this it should be understood that a non-invasive method of imaging can be used along with, at the same time as, or in combination with an invasive procedure, such as surgery.

In some embodiments, the method can be used to diagnose cancer in a subject or detect cancer in a particular organ of a subject. A particularly useful aspect of this method is the ability to search for metastatic cancer cells in secondary tissues or organs, such as lymph nodes, or at or near tumor margins. Therefore, the disclosed methods can be used for assessing lymph node status in patients that have or are suspected of having cancer, such as breast cancer. This may avoid the need to biopsy the tissue or organ, e.g., remove a lymph node. In some embodiments, the method involves administering to the patient the disclosed compounds and detecting whether the compounds have bound to cells in a lymph node. In some of these embodiments, the lymph node can be an axillary lymph node (ALN). In other embodiments, the lymph node can be a sentinel lymph node. In further embodiments, both axillary and sentinel lymph nodes can be assessed for binding of the agent to cells in the lymph node.

The method can also be used with other therapeutic or diagnostic methods. For example, the method can also be used during an operation to, for example, guide cancer removal, which is referred to herein as “intraoperative guidance” or “image guided surgery.” In a particular embodiment, the method can be used for therapeutic treatment to remove or destroy cancer cells in a patient's lymph nodes. For example, the disclosed compounds can be administered to a patient, and the location of cancerous tissue (e.g., lymph nodes) can be determined and removed using image guided surgery. In another preferred embodiment, the method can be used for therapeutic treatment to prevent positive microscopic margins after tumor resection. For example, the disclosed compounds can be administered to a patient, the location of cancer cells around a tumor can be determined, and the complete tumor removed using image guided surgery. In these embodiments, the physician administers the disclosed compounds to the patient and uses an imaging device to detect the cancer cells, guide resection of tissue, and assure that all of the cancer is removed. In addition, the imaging device can be used post-operatively to determine if any cancer remains or reoccurs.

In some embodiments, the disclosed compounds can be linked to a therapeutic compound. The therapeutic compound or moiety can be one that kills or inhibits cancer cells directly (e.g., cisplatin) or it can be one that can kill or inhibit a cancer cell indirectly (e.g., gold nanoparticles that kill or destroy cancer cells when heated using a light source). If the therapeutic compound or moiety is one that kills or inhibits a cancer cell indirectly, then the method further comprises a step of taking appropriate action to “activate” or otherwise implement the anti-cancer activity of the compound or moiety. In a specific embodiment, the therapeutic compound or moiety attached to the agent can be a gold nanoparticle and following administration to the patient and binding of the agent to cancer cells, the gold nanoparticles are heated, e.g., using a laser light, to kill or destroy the nearby cancer cells (photothermal ablation). For example, in some embodiments, the method involves image guided surgery using the disclosed compounds to detect and resect cancer from a subject followed by the use of the same or different disclosed compounds linked to a therapeutic compound to kill remaining cancer cells.

The cancer of the disclosed methods can be any cell in a subject undergoing unregulated growth. The cancer can be any cancer cell capable of metastasis. For example, the cancer can be a sarcoma, lymphoma, leukemia, carcinoma, blastoma, or germ cell tumor. A representative but non-limiting list of cancers that the disclosed compositions can be used to detect include lymphoma, B cell lymphoma, T cell lymphoma, mycosis fungoides, Hodgkin's Disease, myeloid leukemia, multiple myeloma, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, triple negative breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, gliosarcoma, Kaposi sarcoma, esophageal cancer, hepatocellular cancer, and pancreatic cancer.

The cancer can be breast cancer. Breast cancers originating from ducts are known as ductal carcinomas, and those originating from lobules that supply the ducts with milk are known as lobular carcinomas. Common sites of breast cancer metastasis include bone, liver, lung and brain.

The cancer can be non-small-cell lung carcinoma (NSCLC). NSCLC is any type of epithelial lung cancer other than small cell lung carcinoma (SCLC). The most common types of NSCLC are squamous cell carcinoma, large cell carcinoma, and adenocarcinoma, but there are several other types that occur less frequently, and all types can occur in unusual histologic variants and as mixed cell-type combinations.

### Therapeutic Methods

Methods of treating or preventing diseases or disorders are provided using the disclosed compounds. The disclosed compounds can be used for targeting CD206⁺ expressing cells. The disclosed compounds can be used for targeting of macrophages for treatment of intracellular pathogens (M. tuberculosis, F. tularensis, S. typhi). The disclosed compounds can be used to target tumor-associated macrophages, e.g. to be used for treating cancer.

Macrophage-related and other CD206 high expressing cell-related diseases for which the compositions and methods herein may be used include, but are not limited to: acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, histocytosis, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, Rosai-Dorfman disease, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

The disclosed compounds can include therapeutic agents including, but not limited to, cytotoxic agents, anti-angiogenic agents, pro-apoptotic agents, antibiotics, hormones, hormone antagonists, chemokines, drugs, prodrugs, toxins, enzymes, or other agents. The disclosed compounds can include chemotherapeutic agents; antibiotics; immunological adjuvants; compounds useful for treating tuberculosis; steroids; nucleotides; peptides; or proteins, such as those described above.

In certain embodiments, the disclosed compounds include a chemotherapeutic agent for the treatment or prevention of cancer. The cancer can be any cancer cell capable of metastasis. For example, the cancer can be a sarcoma, lymphoma, leukemia, carcinoma, blastoma, or germ cell tumor. A representative but non-limiting list of cancers that the disclosed compositions can be used to treat or prevent include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, triple negative breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, gliosarcoma, Kaposi sarcoma, esophageal cancer, hepatocellular cancer, and pancreatic cancer.

In certain embodiments, the disclosed compounds are effective for treating autoimmune diseases, such as rheumatoid arthritis, lupus (SLE), or vasculitis. In certain embodiments, the disclosed compounds are effective for treating an inflammatory disease, such as Crohn's disease, inflammatory bowel disease, or collagen-vascular diseases.

One of ordinary skill in the art will appreciate that various kinds of molecules and compounds (e.g., therapeutic agents, detection labels, and combinations thereof) can be delivered to a cell or tissue using the disclosed compounds.

In one aspect, provided herein is a method of treating tuberculosis comprising administering to a subject in need thereof a compound as described herein.

In another aspect, provided herein is a method of diagnosing and treating a macrophage-mediated disorder comprising administering to a subject in need thereof an effective amount of a compound as described herein; and detecting the detection label at a predetermined location in the subject.

In another aspect, provided herein is a method of treating a macrophage-mediated disorder comprising administering to a subject in need thereof an effective amount of a compound as described herein.

In another aspect, provided herein is a method of treating a disease comprising administering to a subject in need thereof an effective amount of a compound according as described herein wherein the disease is an autoimmune disease, an inflammatory disease, or cancer.

In another aspect, provided herein is a method of targeting tumor-associated macrophages comprising administering to a subject in need thereof an effective amount of a compound as described herein.

In another aspect, provided herein is a method according to any of those described herein, wherein the compound contains at least one therapeutic agent and at least one detection label.

In another aspect, provided herein is a method according to any of those described herein, wherein a linker is used to attach the one or more CD206 targeting moieties, one or more therapeutic agents, and/or the one or more detection labels.

In another aspect, provided herein is a method according to any of those described herein, wherein the macrophage-mediated disorder is selected from the group consisting of tuberculosis and Leishmaniasis.

In another aspect, provided herein is a method according to any of those described herein, wherein the disease is rheumatoid arthritis.

In another aspect, provided herein is a method according to any of those described herein, wherein the disorder is cancer.

In another aspect, provided herein is a method according to any of those described herein, wherein the cancer is a sarcoma, lymphoma, leukemia, carcinoma, blastoma, melanoma, or germ cell tumor.

In another aspect, provided herein is a method according to any of those described herein, wherein at least one A is a detection label and the detection label is a fluorophore.

In another aspect, provided herein is a method according to any of those described herein, wherein at least one Li-A comprises a chelator.

### ADMINISTRATION

The disclosed compounds can be administered via any suitable method. The disclosed compounds can be administered parenterally into the parenchyma or into the circulation so that the disclosed compounds reach target tissues (e.g., where cancer cells may be located). The disclosed compounds can be administered directly into or adjacent to a tumor mass. The disclosed compounds can be administered intravenously. In still other embodiments, the disclosed compounds can be administered orally, intraperitoneally, intramuscularly, subcutaneously, intracavity, or transdermally.

Parenteral administration of the compounds, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

### GENERAL SYNTHETIC METHODS

Compositions of the present disclosure will now be described by reference to illustrative synthetic schemes for their general preparation below and the specific examples that follow. Artisans will recognize that, to obtain the various compositions herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate.

Chromatography, recrystallization and other conventional separation procedures may also be used with intermediates or final products where it is desired to obtain a particular isomer of a compound or to otherwise purify a product of a reaction.

General methods of preparing compositions described herein are depicted in exemplified methods below.

In some embodiments, the compositions of described herein can be synthesized according to the procedure as shown in Scheme A1.

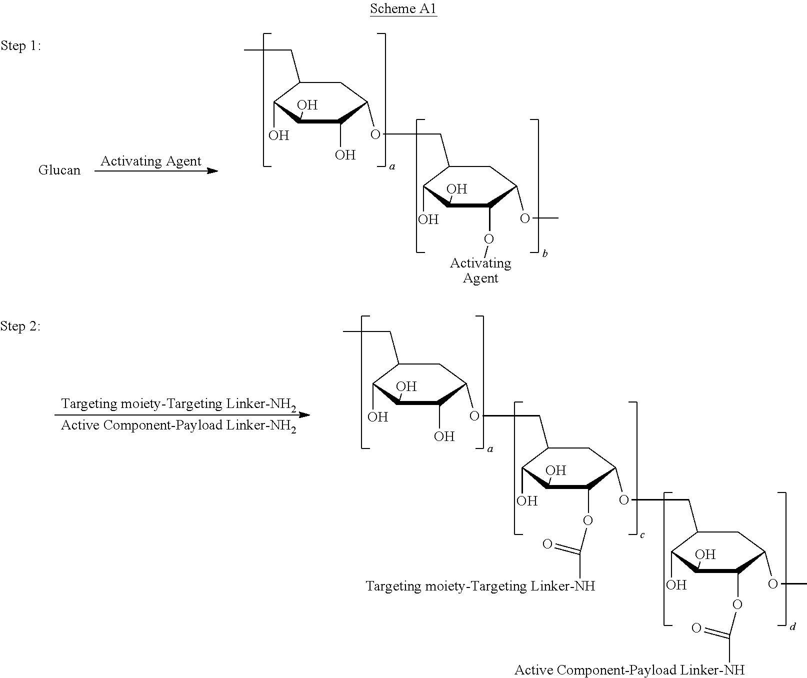

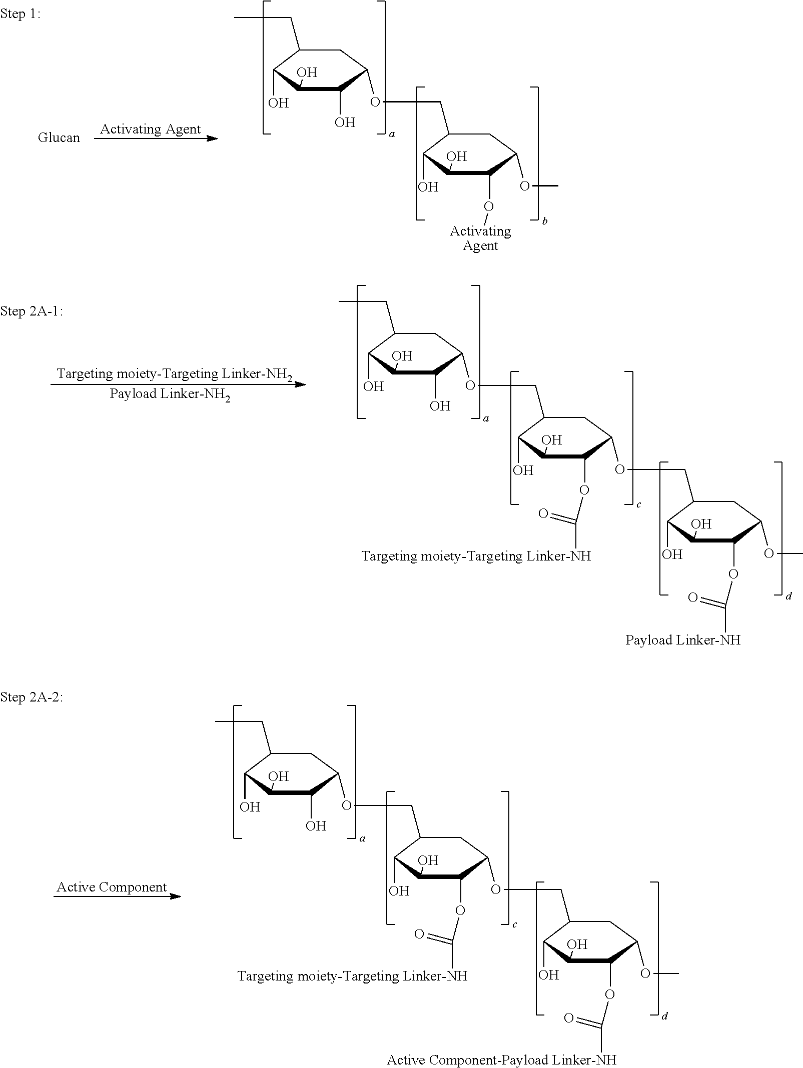

As can be seen in the above schemes, a glucan compound (such as a dextran or a cyclodextrin) is reacted with an activating agent. The resulting activated glucan derivative can then be reacted with the appropriate reagents to introduce a targeting moiety coupled to the glucan backbone via a targeting linker, as well as an active component linked to the glucan backbone via a payload linker. The skilled artisan will recognize that the above schemes are illustrative and that the various reagents and order of synthetic steps can be varied as required for obtaining the intended final products.

### EXAMPLES

The following examples are included for illustrative purposes only and are not intended to limit the scope of the invention.

### Example 1: Structure and Synthesis of Target 3, a Test Compound Conjugated to FITC

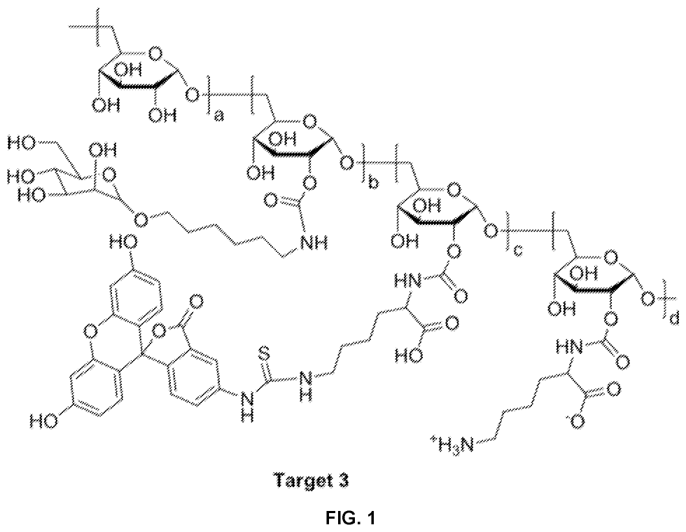

Target 3, shown in FIG. 1, consisted of a mannosylated dextran backbone conjugated to a FITC payload. The attachment of mannose to a dextran backbone serves as a targeting ligand for mannose binding sites, while FITC allows for detection of test compound using confocal or surgical microscopy. The dextran backbone presented here has a molecular weight of about 10 kDa.

### Example 2: Internalization of FITC-Conjugated Drug Target 3 by CD206⁺ Macrophages

A time course endocytosis assay was used to assess macrophage internalization of Target 3, a construct composed of a dextran backbone with mannose as a targeting moiety conjugated to FITC. Resolving whether the test compound was simply bound to the surface or internalized by macrophages was critical in evaluating its potential to reach the desired drug target. Uptake by CD206⁺ macrophages and human embryonic kidney cells (HEK293) (ATCC® CRL-1573™), a cell line lacking CD206 expression, was monitored using confocal microscopy over 34 min. Macrophages and HEK293 cells without antibody and expressing anti-CD206⁺ antibody were included in the assay to determine whether the antibody would block uptake of Target 3.

To prepare plates for the endocytosis assay, a 1 mg/ml stock solution of fibronectin was diluted to 10 μg/mL in PBS without Ca⁺/Mg⁺. Then, 20 μL of the diluted fibronectin solution was added to each well of a 384-well plate (Perkin Elmer LLC CellCarrier™-384 Ultra Microplate). Plate(s) were placed on a level surface at RT for 60 min before excess fibronectin solution was aspirated. Fibronectin-coated plate(s) were used immediately or allowed to air-dry under a laminar flow bench and stored at 4° C. for up to 2 weeks.

Harvested macrophages and HEK293 cells were diluted to a density of 160,000 cells/mL (4,000 cells/well in 25 μL), in their respective growth media. Cytokines and LPS were added to

Complete M1-Macrophage Generation Medium DXF. Cells (25 μL) were added to desired wells and allowed to adhere overnight.

The following morning, test compound Target 3 was diluted in DMSO and added to desired wells with an Echo 555 Liquid Handler, using a ten point three-fold dilution series at a top final concentration of 50 μM. Specific wells were treated only with DMSO. Immediately after addition test compound, the nuclear stain Hoechst was added to all plate wells at a final concentration of 1 μg/mL in a final volume of 50 μL. Cells were then incubated for 10 min at 37° C. in a humidified incubator with 5% CO₂.

Plate wells were imaged with the Opera Phenix™ High Content Screening System using confocal imaging with a 20X water objective, 9 fields per well, and the Hoechst and Alexa 488 filters. Wells were imaged at 10, 20, and 34 min after addition of Hoechst.

Images were analyzed with the Columbus Image Data Storage and Analysis System to generate quantitative measures of compound fluorescent intensity in the nucleus and cytoplasm of macrophages and HEK293 cells, and to identify the number of cells with compound fluorescent intensity above background levels, in this case ≥20,000 RFUs. Microsoft Excel was used to compare quantitative data for macrophages and HEK293 cells in graphic format.

The percentage of internalization of Target 3 was detected after incubation with 50 μM Target 3. Nearly 100% of macrophages with and without antibody internalized Target 3 after 10 min, indicating that Target 3 reached the desired drug target and the anti-CD206⁺ antibody did not interfere with compound uptake. This mean percentage of cell uptake was maintained throughout the course of the assay, 34 min. In comparison, uptake of Target 3 by both groups of HEK293 cells was <26% at 34 min.

For reference, uptake of the 10,000 MW dextran pHrodo™ green was also evaluated in macrophages and HEK293 cells. Allowing for resolution between binding and cellular uptake, the pHrodo™ green dextran fluoresces strongly in acidic conditions but is relatively non-fluorescent at a neutral pH. Internalization of pHrodo™ green increased over time, reaching 90% uptake at 17 h. Uptake by HEK293 cells was undetectable at other time points but reached 14% at 17 h. Compared to macrophage internalization of Target 3 after 10 min, 17 hours passed before a similar percentage of human macrophages internalized pHrodo™ green.

### Example 3: Structure and Synthesis of Target-5, a Targeted Chemotherapeutic Composed of a Mannosylated Dextran Ligand Connected by a Valine-Citrulline Linker to the Toxin Monomethyl Auristatin E

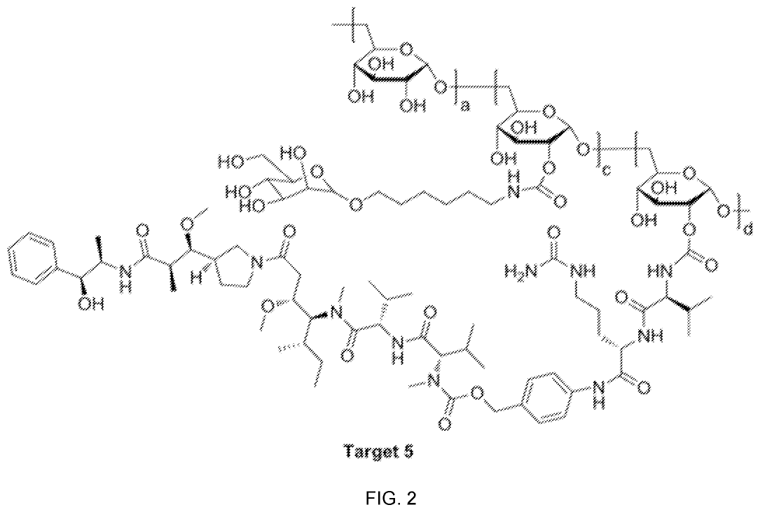

Target-5 consists of four components (ABCD). To form a mannose binding site targeting moiety, a dextran backbone (A) was mannosylated (B). The A and B components that make up the targeting ligand are connected by a valine-citrulline linker (C) to a toxin (D). Here, the linker joins the toxin monomethyl auristatin E (MMAE) to the targeting moiety. A representative Target 5 molecule is shown in FIG. 2.

### Example 4: Reduction of U87-MG Tumor Volume in vivo After Treatment with Target-5

The anti-tumor activity of Target-5, a chemotherapeutic construct composed of a mannosylated dextran backbone connected to the toxin monomethyl auristatin E (MMAE) with a valine-citrulline linker, was assessed in vivo using a mouse model of glioblastoma. Three distinct doses of Target-5 were evaluated against temozolomide, an FDA-approved chemotherapeutic for the treatment of glioblastoma, and a negative control in athymic nude mice bearing U87-MG tumors.

To provide a murine glioblastoma model, U87-MG (ATCC® HTB-14™) cells were injected into the crania of outbred athymic nude mice (Jackson Laboratories) from 4-6 weeks of age. In preparation for intracranial injection, U87-MG cells were grown for 10-14 days in Fetal Bovine Serum supplemented with Eagle's Minimum Essential Medium (EMEM)+1X Penicillin/Streptomycin, then split 1:5 upon reaching confluency. For approximately 3-4 min at 37° C., cells were harvested from tissue culture flasks at approximately 70% confluency with 3.0 ml of Tryp LE Express per flask. Trypsin activity was halted by adding 8 mls of complete media to each 75 cm 2 flask, and detached cells were collected with a sterile 10 ml stripette. Cells were centrifuged for 4 min at 4° C. at 1,100 RPMs, supernatant was aspirated, and cells were washed twice with sterile 1X PBS containing cations. Cells were then resuspended in 1X PBS. A Hamilton syringe was used to intracranially inject a 5 μl volume containing 500,000 cells per brain.

The surface area of a ventilated Animal Transfer Station (ATS) was used as the surgical area. The ATS surface was sterilized with 70% ethanol prior to placing the KOPF stereotaxic apparatus and surgical instruments on its surface. Mice were anesthetized in preparation for surgery. Mice bellies were swabbed with ethanol before a 40 μl intraperitoneal injection of a Ketamine-Xylazine mixture in sterile saline. Once anesthetized, the scalp was prepared by swabbing it with a sterile alcohol prep pad (70% isopropyl alcohol). Eye ointment was applied to both eyes in order to maintain moisture during the procedure. Using a sterile scalpel, a sagittal incision of approximately 1 cm long was performed over the head. The exposed skull surface was then cleaned and dried using a sterile cotton swab applicator. Once the cranial bones dried, the bregma became visible.

For intracerebral tumor establishment, a sterile 25-gauge sharp needle was used to puncture the skull to create a small hole in the cranium for the subsequent injection of tumor cells. Cells were injected into the brain at coordinates starting 3 mm right of the bregma, 1 mm anterior of the coronal suture, and 3 mm deep from the surface of the cerebral cortex. The needle was brought down 3.5 mm from the surface to minimize the reflux of cells during the injection and to create a small pocket so that most of the injected cells stay 3 mm from the brain surface. The syringe was placed perpendicular to the skull, over the previously created cranial hole, then lowered. The cell suspension was slowly injected at an approximate rate of 1 μl to 1.5 μl per min. The needle was kept in place for another minute before slow withdrawal to reduce reflux of the injected tumor cells.

The skull was cleaned and dried using a sterile dry cotton swab. Using sterile forceps, the scalp was drawn together over the skull and tissue glue was added to the incision. The scalp was then cleaned, and a triple antibiotic ointment was applied over the incision. Post-operatively, mice were monitored until they woke up from the anesthesia and normal activity was recovered.

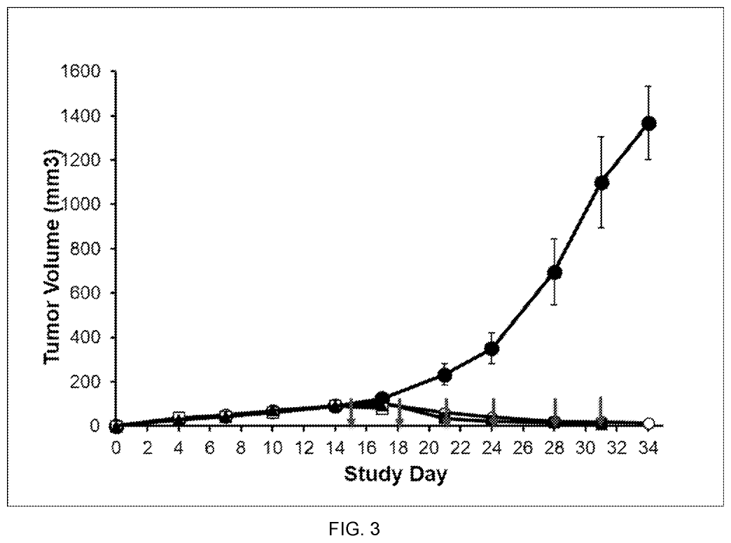

As shown in FIG. 3, treatment with Target-5 resulted in significant reduction in tumor volume (mm³) compared to mice treated with vehicle. Tumors removed from mice treated with vehicle (A), 5 mg/kg Target-5 (B), and 50 mg/kg Target-5 (C) are pictured in FIG. 3. The data suggest dose-dependent anti-tumor activity of Target-5. A 10-fold increase in the dose of Target-resulted in a 2-fold increase in anti-tumor activity. The results of this study show that anti-tumor efficacy of Target-5 was comparable to that of the standard chemotherapeutic used to treat glioblastoma, temozolomide.

### Example 5: Structure and Synthesis of Target-6, a Modified Cyclodextrin That Facilitates Intra-Operative Imaging of the Brain-Tumor Parenchyma Neovascular Network and Targeted Chemotherapeutic Delivery

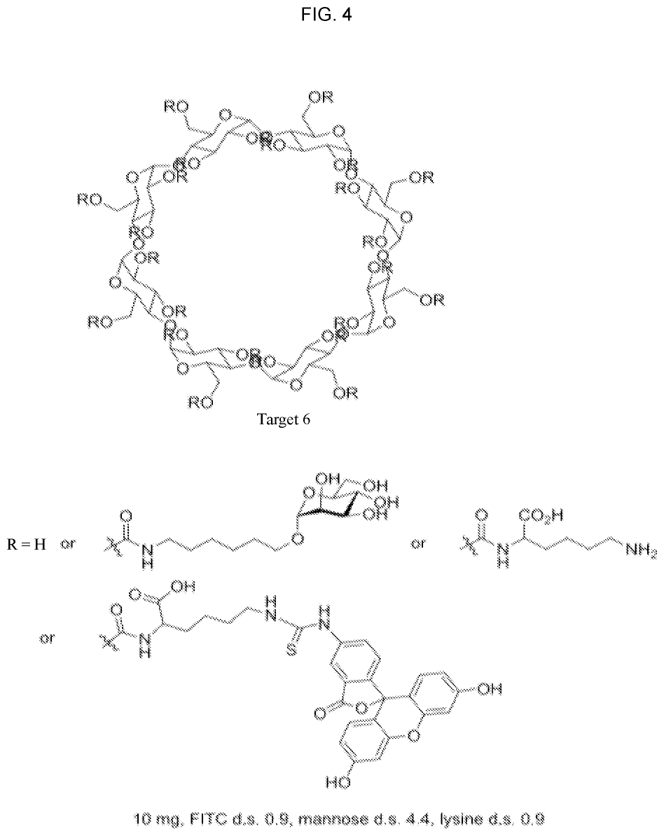

Target-6, as shown in FIG. 4, is composed of a cyclodextrin backbone, mannose as a targeting moiety, and lysine for tissue fixation. Conjugated to FITC or another fluorescent moiety, Target-6 offers utility as an intra-operative imaging agent by allowing for accurate and specific visualization of the brain-tumor parenchyma neovascular network. The construct could further be useful as a targeted chemotherapeutic by trading the fluorescent moiety for a linker and a toxin. Importantly, Target-6, with the different shaped backbone, is still able to crosses the blood-tumor barrier. Target-6 is able to carry payloads into brain tumors and across the blood-tumor barrier without leaking across the blood-brain barrier.

### Example 6: Intravenously Injected Fluorescent Target-6 Cyclodextrin Compound Targets the Brain-Tumor Parenchyma Neovascular Network

Target-6, a FITC-labeled cyclodextrin modified with mannose and lysine, was evaluated in vivo to determine whether it targets the brain tumor parenchymal neovascular network. This network is composed of tumor-associated macrophage vascular mimicry, the target of the modified cyclodextrin construct. The potential of Target-6 as an intra-operative imaging agent was assessed by the extent of detection of the compound in the parenchyma. This further served as a surrogate for evaluation of the utility of the construct as a site-specific drug delivery agent, provided replacement of FITC with a cytotoxic compound. The specificity of Target-6 and time from injection to detection were important to determining the utility of the construct as an intra-operative imaging agent.

To evaluate its utility in vivo, U87-MG tumor cells were implanted as described above and, after 10-12 days, Target-6 was intravenously injected at 50 mg/ml (200-250 μl) into the tail vein of the athymic nude mice and allowed to circulate. Images were taken at 10-12 days after implantation and initial administration. The compound was allowed to circulate systemically for either 2 or 3 min before mice were euthanized with isoflurane followed by cervical dislocation.

Brains were then harvested. Harvested brains were fixed overnight in 4% PFA/PBS at 4° C. The next morning, the brains were rinsed with 4 mls of 1X PBS then rested overnight in a 15% sucrose solution at 4° C. The following morning, the brains were transferred to a 35% sucrose solution in which they were stored overnight at 4° C. Brains were frozen in optimal cutting temperature compound and sectioned on cryostat at 60 micron thickness.

Sections were washed 3X with PBS then nuclei were stained with Hoechst 33342 for 15-20 min at RT in darkness. Sections were washed 3X with PBS and mounted on poly-L-Lysine-coated frosted slides with a drop of slowfade reagent. Appropriate no-secondary controls were performed in all experiments.

All images were gathered with a confocal laser-scanning microscope (LSM 700 or 710, Carl Zeiss) utilizing a Plan-Apochromat 20X/0 8, Plan-Apochromat 63X/I 4 Oil DIC, CApochromat 40X/1 2W Korr UV-VIS objective lens (Carl Zeiss) and processed with the ZEN 2010 software (Carl Zeiss). Scanning was performed in sequential laser emission mode to avoid scanning at other wavelengths. Three-dimensional reconstructions were generated using ZEN 2010. Z-stacks were acquired using a Zeiss 710 laser scanning confocal microscope using a 20X objective (1 μm step size), or a 63X objective (0.3 μm step size) and assembled in the Zen software (4 experiments, n=3-5 per experiment).

Detection of Target-6 post-injection was performed by imaging brains treated with Hoechst nuclear stain in the blue fluorescent channel. Target-6 labeled with FITC targeted the brain-tumor parenchyma neovascular network, indicative of its potential utility as an intra-operative agent. Visualization specific to a tumor, without distortion from off-target imaging of surrounding tissue, is vital to determining the size and location of said tumor.

In addition, the near immediate localization to tumor as well as extended residence in tumor tissue (24 hour) gave a wide window for surgery. Fluorescein is time sensitive, sometime the dye is washed out when the surgeon gets down to the tumor or else the dye is not tumor specific with leak given its low molecular weight.

Regarding its potential as a therapeutic, the sequestration into tumor tissue of Target-6 could be exploited by replacing FITC with a cytotoxic compound. Targeted delivery of the cytotoxic agent to tumor tissue using Target-6 would reduce delivery to surrounding normal brain tissue, and thereby decrease off-target toxicity.

For reference, fluorescein (FITC) alone was intravenously injected into athymic mice bearing U87-MG tumors. FITC showed little tumor specificity after 5 min of systemic circulation. Two hours post-intravenous injection, FITC had substantially washed out from the tumor and surrounding tissue. The lack of specificity of FITC for tumors in mice compared to the specificity of the molecules disclosed here clearly demonstrates an improvement in the delivery of FITC for intra-operative imaging.

### Example 7: Synthesis of Target-7, a Targeted Magnetic Resonance Imaging Agent Comprising DOTA and Gadolinium

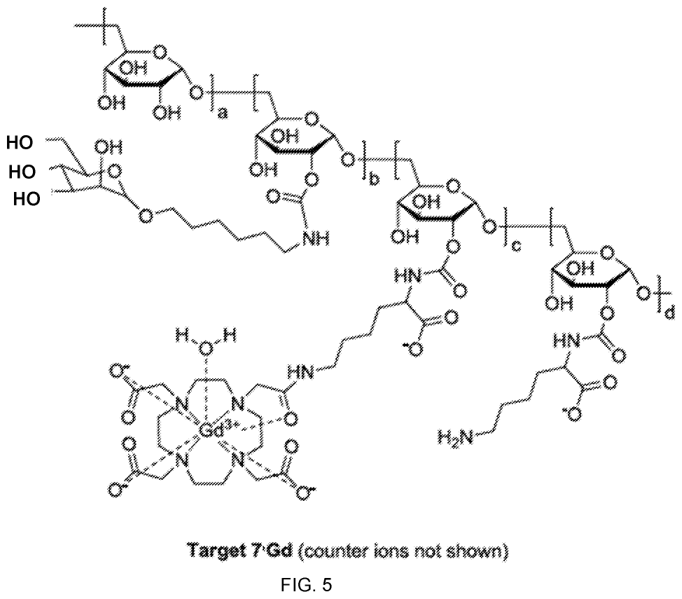

A gadolinium-labeled construct comprised of a targeting element, a dextran backbone, and a DOTA chelator was synthesized to produce a compound with specificity for tumor-associated macrophages, capable of detection using MRI. An exemplary molecule is shown in FIG. 5.

### Example 8: Synthesis of a Targeted Radiotherapeutic Comprising DOTA and Lutetium-177

A lutetium-177-labeled construct similar to that shown in FIG. 5 comprised of a tumor-associated macrophage-targeting element, a dextran backbone, and a DOTA chelator was synthesized to produce a radiotherapeutic with activity against solid and metastatic tumors.

Following MRI-detection of Target-7 to determine the size and location of a primary tumor and metastasized cancer cells, radiotherapy utilizing the targeted lutetium-labeled construct would be provided. Subsequent administration of Target-7 would allow for assessment of radiotherapeutic efficacy on the size of a tumor or tumors and the extent of metastasized cells.

### Example 9: Reduction of 4T1 Triple Negative Breast Cancer Tumor Volume in vivo After Treatment with Target-5

The anti-tumor activity of Target-5 was assessed in vivo using a mouse model of triple negative breast cancer. Two distinct doses of Target-5 were evaluated against paclitaxel, an FDA-approved chemotherapeutic agent, and a vehicle control in BALB/c mice.

Specifically, six-week-old female BALB/c mice (Charles River Laboratories) were inoculated with 1×10⁵ 4T1 triple negative breast cancer cells per animal in the 3^{rd} mammary fat pad region on Study Day 0. Randomization was performed; tumor volumes averaged 162 mm 3 and the weight ranged from 17.6-18.4 gm/mouse (n=10/group) at start of the study.

Target-5 was formulated in 0.9% saline and the mice were treated twice a week with either 5 mg/kg or 15 mg/kg Target-5 by tail vein injection. The dosing volume was adjusted for body weight. Paclitaxel was administered at a dosage of 15 mg/kg and given twice weekly intravenously.

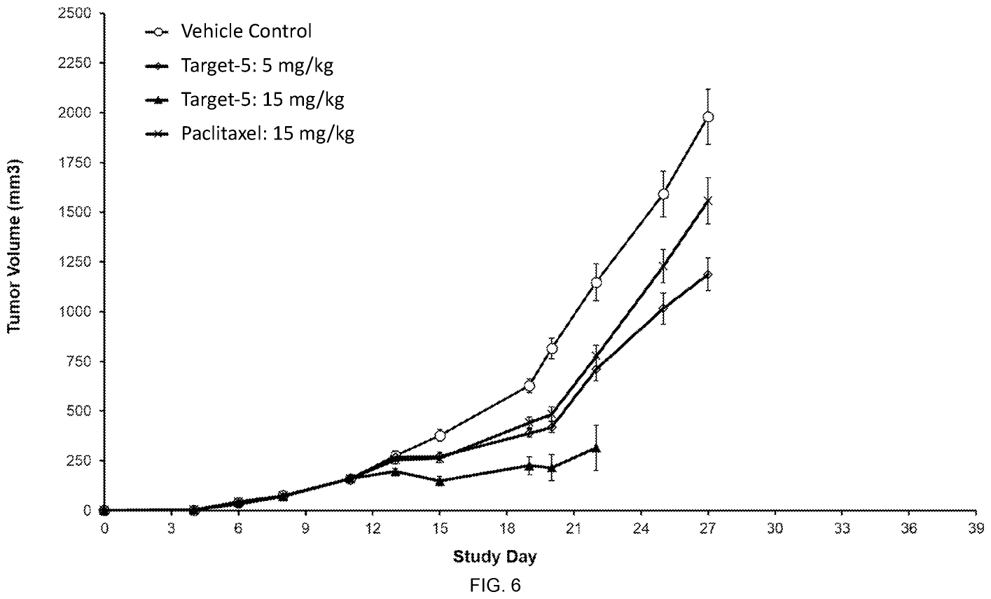

As shown in FIG. 6, treatment with Target-5 resulted in significant reduction in tumor volume (mm³) compared to mice treated with vehicle. Mice treated with either dosage of Target-5 also experienced reduced tumor volume as compared to mice treated with paclitaxel with mice receiving 15 mg/kg of Target-5 experiencing the lowest tumor burden. These data suggest dose-dependent anti-tumor activity of Target-5. Additionally, these data suggest that the anti-tumor efficacy of Target-5 was more effective at reducing tumor volume that paclitaxel in this 4T1 triple negative breast cancer model.

### Example 10: Target-5 Extends Survival in U87 Intracranial Model of Glioblastoma

To determine survival rates in mice treated with Target-5, mice were implanted with an intracranial tumor. Specifically, at study day 0, all mice were inoculated intracranially with U-87 MG cells (at 0.5×10⁶ cells/animal). U87MG cells had been cultured in DMEM/10% FBS.

The surgeries were performed on the sterilized surface area of a ventilated Animal Transfer Station. Mice were anesthetized with 1.5-2% isoflurane. Once anesthetized, the scalp was swabbed with sterile alcohol prep pad. Puralube Vet eye ointment was applied to both eyes. Using a sterile scalpel, an approximately 1 cm long sagittal incision was made down the center of the head to expose the skull. The skull was cleaned and dried using a sterile cotton swab applicator allowing visualization of the bregma. A burr hole through the skull was made using a sterile 25-gauge needle at stereotactic coordinates and 0.5×10⁶ U87MG tumor cells were injected in a 5 μl volume using the following coordinates (3 mm right of the bregma, 1 mm anterior to the coronal suture, and 3 mm deep. The needle was introduced to a 3.5 mm depth and then retracted 0.5 mm to create a pocket to minimize the reflux of cells during the injection. The cell suspension was resuspended prior to each cell implantation and the cells were slowly injected at an approximate rate of 1 μl to 1.5 μl per min. The needle was kept in place for another minute before slow withdrawal to reduce reflux of the injected tumor cells. Following tumor cell implantation, the skull was cleaned and dried using a sterile dry cotton swab. Using sterile forceps, the incision was closed with tissue glue. The scalp was cleaned, and a triple antibiotic ointment was applied over the incision. Post-operative buprenorphine-SR was used as an analgesic at 1 mg/kg (1 mL/kg). Mice were monitored postoperatively, in a warm cage (using circulating water heating pad) until they resumed normal activity.

At study day 9, body weights were measured for randomization, and mice were stratified by weight into 3 groups of 10 animals to obtain similar average body weight among groups. After randomization, test article administration (saline or Target-5) was started.

Test articles were administered to each animal based on individual body weight. At study day 0, the mean weight of mice in each group was 24.7 gm. Test articles were administered intravenously into the lateral tail vein or orally twice a week for 45 days. Drugs were formulated fresh for each treatment.

Animals were weighed three times per week. Weight loss (in excess of 20% compared to Day 0) would result in euthanasia. If weight loss -10% is observed, animals will be provided with daily subcutaneous dose of 0.1 mL saline, powdered and moistened food on petri dish and hydrogel in the cage. If necessary, mice will be given 0.1 mL PO.

Mice were checked daily for signs of distress and if meeting criteria per IACUC guidelines, were euthanized. On Study Day 45 all remaining mice were euthanized by isoflurane overdose. Survival Data was analyzed by Prism software.

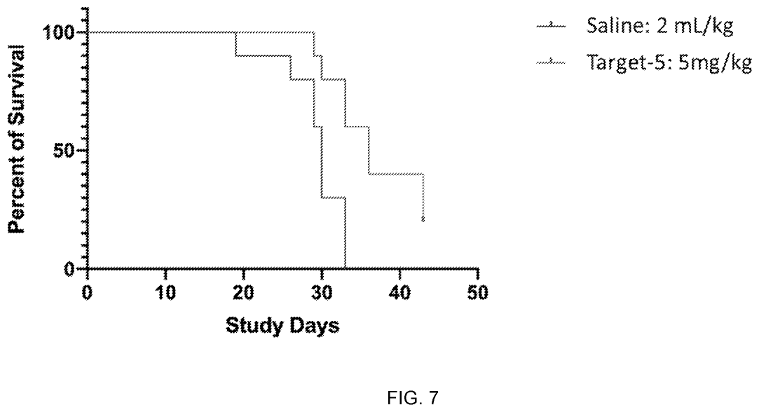

As shown in FIG. 7, mice that were administered Target-5 (5 mg/kg) had a higher percentage of survival than mice that received saline. These data suggest that not only can Target-5 inhibit tumor volume, but Target-5 increases survival rate in an intracranial model of glioblastoma.

### Example 11: Reduction of Glioma Tumor Volume in vivo After Treatment with Target-5

The anti-tumor activity of Target-5 was assessed in vivo using an immunocompetent glioma mouse model. Three distinct doses of Target-5 were evaluated against Temozolomide, an FDA-approved chemotherapeutic agent, and a vehicle control in C57BL/6 mice.

Specifically, female C57BL/6 mice (Jackson Laboratories) were inoculated with 5×10⁶ GL261 cells (mycoplasma tested-negative) with 96% viability and 100% tumor take rate (cell passages prior to plating (#7)). Tumors were allowed to grow until they reached an average tumor volume of 95.1 mm3 and mice had an average body weight of 21.0 grams.

Target-5 was formulated in 0.9% saline and the mice were treated twice a week with either 5 mg/kg, 7.5 mg/kg, or 10 mg/kg Target-5 by tail vein injection. The dosing volume was adjusted for body weight. Temozolomide was formulated in 10% DMSO in 0.9% sodium chloride and mice were treated twice a week by gavage at a dosage of 12.5 mg/kg. n=10 for each group.

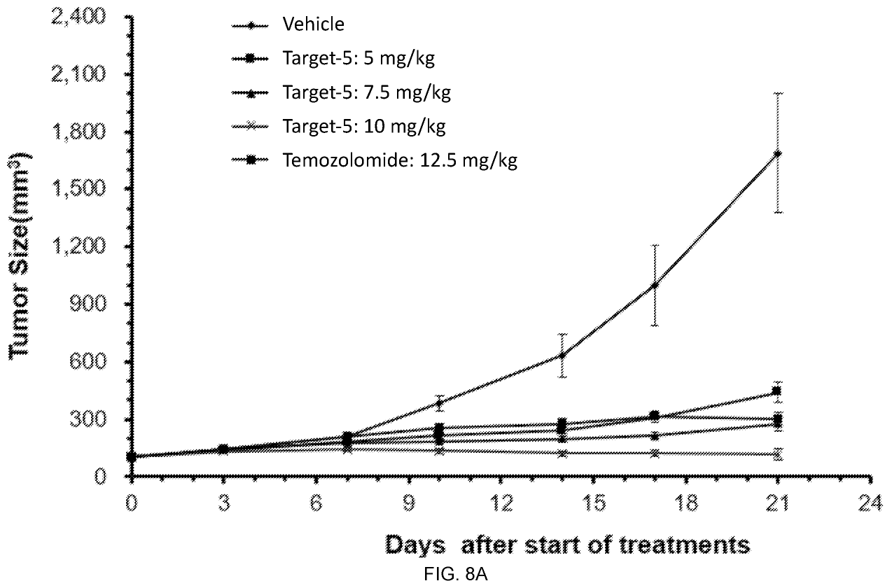

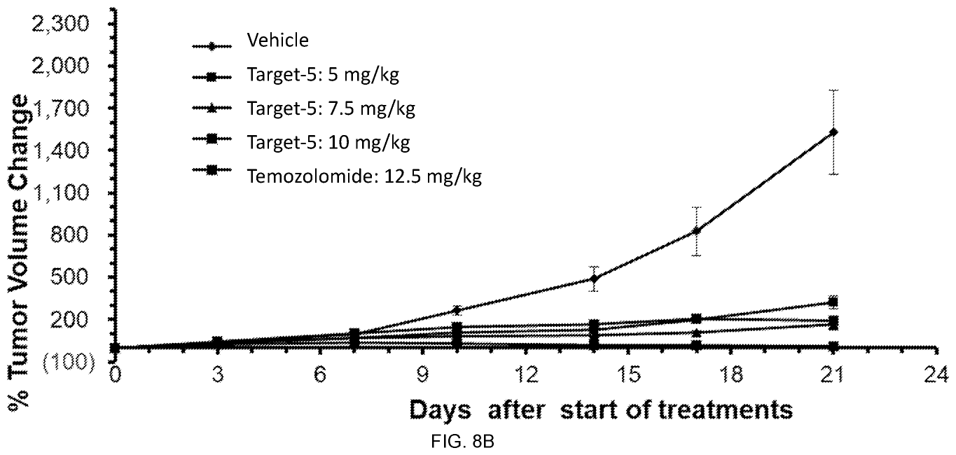

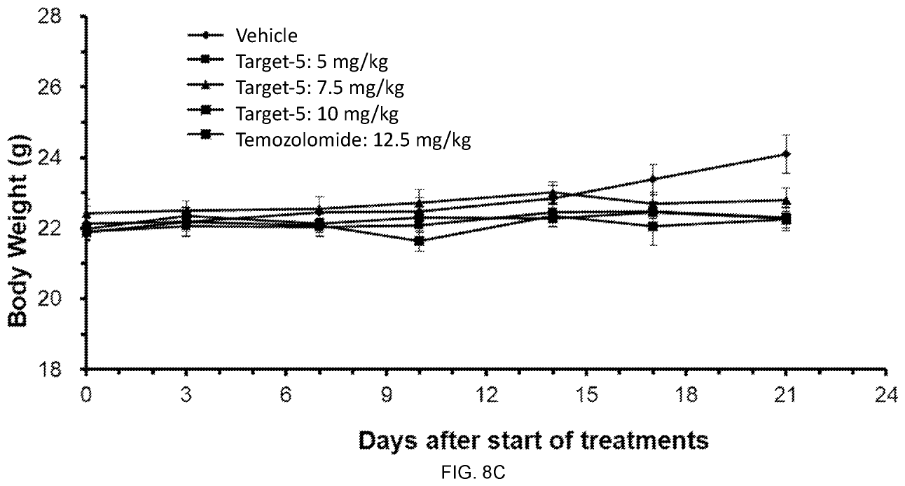

As shown in FIG. 8A and 8B, treatment with Target-5 inhibited tumor volume (mm³) in a dose-dependent fashion as compared to mice treated with vehicle. On day 21, mean tumor volume vehicle-treated=1687 mm³, SD=928.9 mm³; Mean tumor volume 5 mg/kg Target 5 treated=440.5 mm³, SD=159.2 mm³; Mean tumor volume 7.5 mg/kg Target 5-treated=275.1 mm³, SD=120.7 mm³; Mean tumor volume 10 mg/kg Target 5-treated=117.2 mm³, SD=89.6 mm³; Mean tumor volume temozolomide-treated=302.6 mm³; SD=99.7. These data show that the protection provided by Target-5 is greater than the protection provided by temozolomide in this immunocompetent mouse glioma model. While Target-5 and temozolomide inhibited tumor volume progression, there was no significant change in the body weight of the mice in any treatment group (FIG. 8C)

### Example 12: Reduction of MC38 Colon Cancer Tumor Volume in vivo After Treatment with Target-5

The anti-tumor activity of Target-5 was assessed in vivo using a mouse model of colon cancer. Two distinct doses of Target-5 were evaluated against Gemcitabine, an FDA-approved chemotherapeutic agent, and a vehicle control in C57BL/6 mice.

Specifically, 8- to 12-week-old C57BL/6 female mice (Charles River Laboratories) were inoculated with 5×10⁵ MC38 tumor cells in 0% Matrigel subcutaneously in the flank in a volume of 0.1 mL/mouse on Study Day 0. A pair match was performed when the tumors reached an average size of 80-120 mm 3 at which time treatment began.

Target-5 was formulated in 0.9% saline and the mice were treated intravenously twice a week with either 5 mg/kg or 10 mg/kg Target-5 and then delivered intraperitoneally after a dosing holiday on day 15. The dosing volume was adjusted for body weight. Gemcitabine was administered at a dosage of 40 mg/kg and delivered intraperitoneally q3 days×4. Tumor was measured by calipers twice a week. Body weight was measured every day for 5 days and then bi-weekly to the end of the study. (n=10/group). The endpoint of study was when tumor volume reached 1500 mm³.

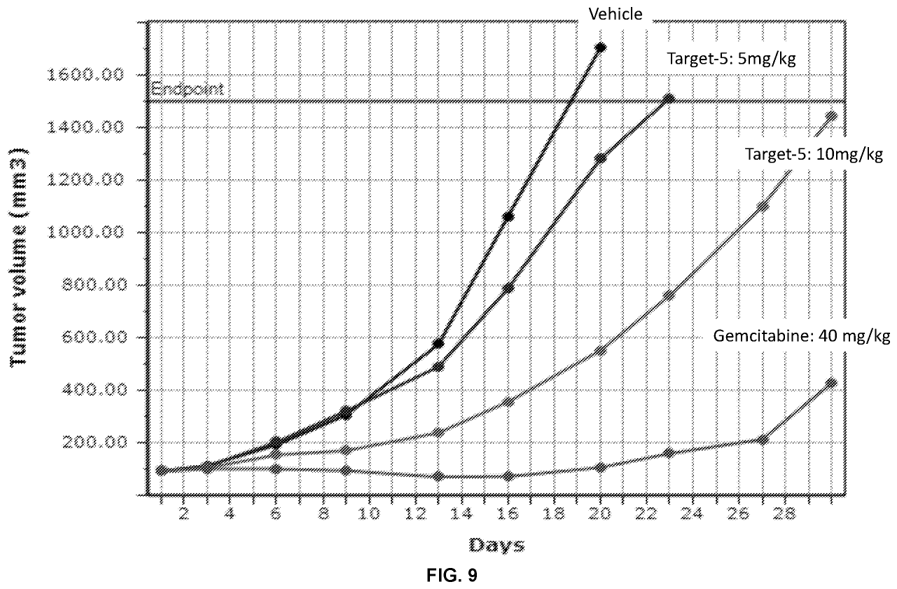

As shown in FIG. 9, treatment with Target-5 resulted in the reduction of tumor volume (mm³) compared to mice treated with vehicle. Gemcitabine appeared to reduce tumor volume more than mice treated with either dosage of Target-5, however multiple doses of Target-5 were missed due to mouse tail swelling resulting in the switching of administration routes from intravenous to intraperitoneally. The data show that mice receiving 10 mg/kg of Target-5 experienced a lower tumor volume than mice receiving 5 mg/kg of Target-5 which suggest dose-dependent anti-tumor activity of Target-5 in this model.

### Example 13: Target-7 Shows Reliable Enhancement of Both Intracranial and Subcutaneous U87MG Tumors

Data was generated comparing Target-7 versus Magnevist, a standard of care gadolinium MRI contrast agent, in both intracranial and subcutaneously implanted U87MG tumors in nu/nu mice. The intracranial model protocol is as described above. Specifically, 50,000 U87 cells were implanted at same coordinates as described previously (n=6). For the subcutaneous tumor model, 4×10⁶ cells were injected into the right flank in a volume of 50 ul (n=6).

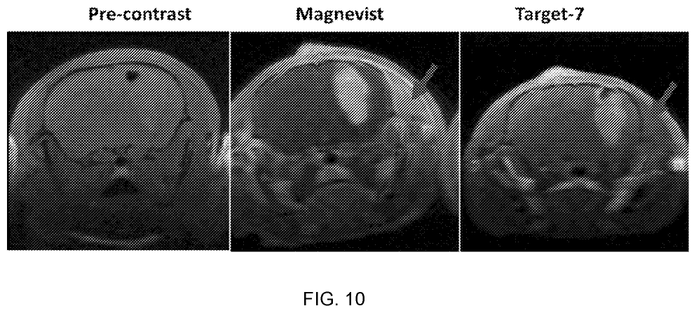

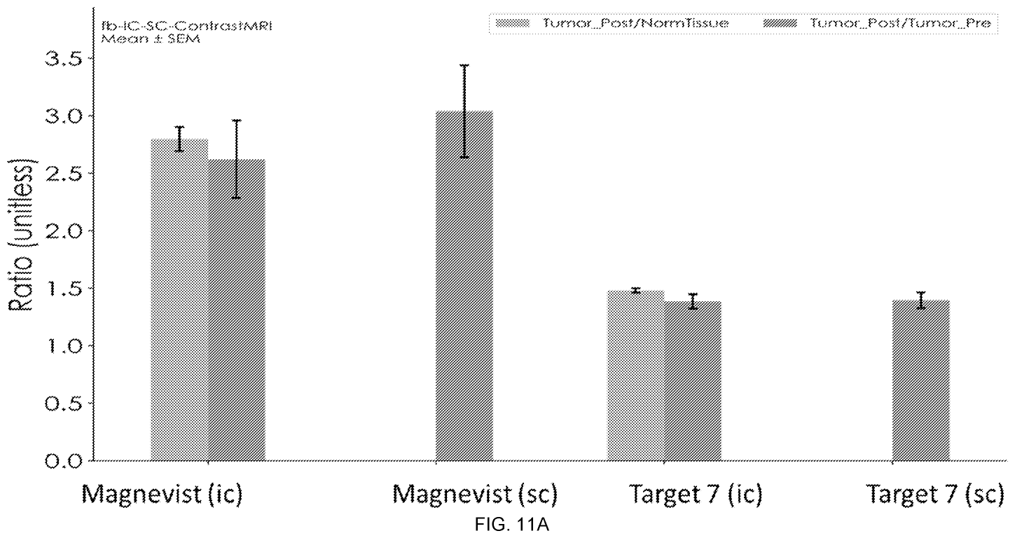

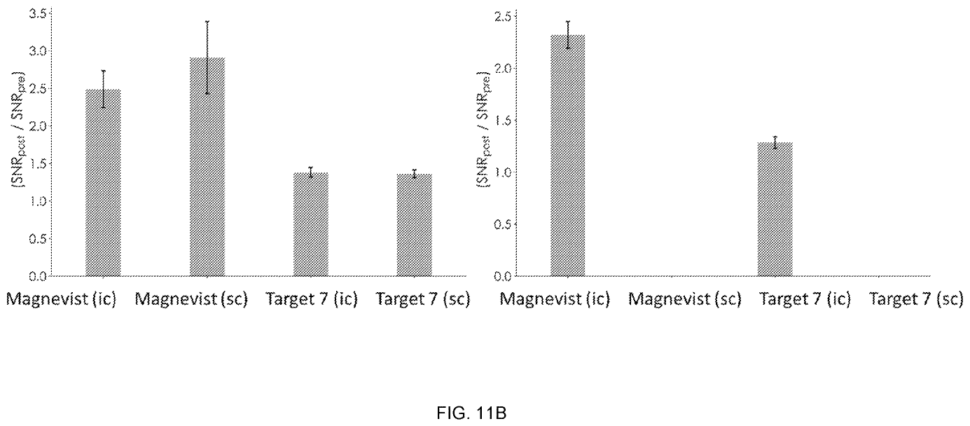

MRI imaging was performed with staggered acquisition between day 14 and 18 post-implantation of tumor cells (FIG. 10). The images illustrate that Target-7 crosses the blood-tumor barrier but does not cross the blood-brain barrier. Target-7 also shows less leakage into normal tissues than Magnevist. For intracranial tumors, the mice were imaged with T2-weighted pre-contrast and T1-weighted (pre and post-contrast administration). In the subcutaneous model, mice were imaged with T1-weighted sequence both pre and post contrast administration. FIG. 11A shows the signal intensity ratios of post-contrast tumor to selected tissues.

A pre-contrast region of interest (ROI) was determined for the subcutaneous tumors and compared with the post-contrast ROI. For the intracranial study, the tumor was contoured as ROI and the contralateral hemisphere was used as a comparator. ROI analysis was performed using VivoQuant Software. The tumor ROI was manually segmented for each transverse slice on the following scans: T1 RARE pre-contrast (all subjects), T1 RARE post-contrast (all subjects), T2.

For mice injected subcutaneously, the Noise ROIs were generated by placing fixed-volume cylinders outside of the animal, but within the field of view (FOV) for all scans listed above. For mice injected intracranially, the Noise ROIs were represented as manually drawn prisms with similar volumes. For mice injected intracranially, the Normal Tissue ROI was generated using a reflection of the Tumor ROI in an area of the brain that did not contain tumor tissue. FIG. 11B shows signal to noise ratios (SNR) of Ti tumor post-contrast to Ti tumor pre-contrast. The graph on the left shows “Noise” defined as intensity of background region containing no tissue in the FOV. The graph on the right shows “Noise” defined as intensity of brain tissue that does not contain tumor. MRI-based Tumor volume was calculated by multiplying the area of each segmented slice by the slice thickness.

1. A composition comprising:
a CD206 targeting moiety coupled to a glucan backbone comprising a plurality of backbone monomers via a targeting linker comprising a carbamate group and a chain moiety, wherein the carbamate group is connected to a backbone monomer and the chain moiety connects the carbamate group and the CD206 targeting moiety, and an active component coupled to the glucan backbone.

2. The composition of claim 1, wherein the plurality of backbone monomers comprises a plurality of D-glucose monomers in a α-1,6 glycosidic linkage.

3. The composition of claim 2, wherein the plurality of D-glucose monomers is n, wherein n=16 to 111.

4. The composition of claim 3, wherein the plurality of D-glucose monomers is n, wherein n=50 to 65.

5. The composition of claim 2, wherein the glucan backbone is a linear dextran molecule.

6. The composition of claim 2, wherein the glucan backbone is a cyclodextrin molecule and n=6 to 16 D-glucose monomers

7. The composition of claim 1, wherein the CD206 ligand comprises at least a portion of mannose, galactose, collagen, fucose, sulfated N-acetylgalactosamine, N-acetylglucosamine, luteinizing hormone, thyroid stimulating hormone, or a chondroitin sulfate.

8. The composition of claim 7, wherein the targeting moiety is a mannose.

9. The composition of claim 8, wherein the ratio of mannose to backbone monomers is about 1 to 5 to about 1 to 25.

10. The composition of claim 7, wherein the ratio of mannose to backbone monomers is about 1 to 6 to about 1 to 19.

11. The composition of claim 7, wherein the degree of substitution of mannose on a cyclodextrin ranges from about 0.1 to about 7.

12. The composition of claim 11, wherein the degree of substitution of mannose on a cyclodextrin ranges from about 0.5 to 5.

13. The composition of claim 1, wherein the targeting linker is connected to the glucan backbone through the oxygen atom of the carbamate group.

14. The composition of claim 1, wherein the chain moiety of the targeting linker comprises a C₃-C₇ alkylene chain.

15. The composition of claim 1, wherein the chain moiety of the targeting linker comprises a C₆-alkylene moiety.

16. The composition of claim 1, wherein the chain moiety of the targeting linker is a unsubstituted C₆-alkylene moiety.

17. The composition of claim 1, wherein the carbon atom of the carbamate group of the targeting linker is the only sp2-hybridized carbon when said linker is attached to mannose.

18. The composition of claim 1, wherein the active component is a detectable marker or a therapeutic agent.

19. The compound of claim 18, wherein the detectable marker is a radioisotope, a metal chelator, an enzyme, a fluorescent compound, a bioluminescent compound, or a chemiluminescent compound.

20. The compound of claim 19, wherein the radioisotope is selected from the group consisting of ²¹²Bi, ¹³¹I, ¹¹¹In, ⁹⁰Y, ¹⁸⁶Re, ²¹¹At, ¹²⁵I, ¹⁸⁸Re, ¹⁵³Sm, 213Bi, ³²P, and ¹⁷⁷Lu.

21. The compound of claim 18, wherein the detectable marker is an imaging agent.

22. The compound of claim 21, wherein the imaging agent is 5-carboxyfluorescein, fluorescein- 5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, DyLight650, IRDye650, IRDye680, DyLight750, Alexa Fluor 647, Alexa Fluor 750, IR800CW, ICG, Green Fluorescent Protein, EBFP, EBFP2, Azurite, mKalamal, ECFP, Cerulean, CyPet, YFP, Citrine, Venus, YPet, a gadolinium chelate, an iron oxide particle, a super paramagnetic iron oxide particle, an ultra-small paramagnetic particle, a manganese chelate, gallium containing agent, 64Cu diacetyl-bis(N4-methylthiosemicarbazone), 18F-fluorodeoxyglucose, 18F-fluoride, 3′-deoxy-3′-[18F]fluorothymidine, 18F-fluoromisonidazole, technetium-99m, thallium, iodine, barium-sulphate, or a combination thereof.

23. The composition of claim 18, where the therapeutic agent is a cytotoxic agent.

24. The compound of claim 23, wherein the cytotoxic agent is selected from the group consisting of ricin, ricin A-chain, doxorubicin, daunorubicin, a maytansinoid, taxol, ethidium bromide, mitomycin, etoposide, tenopo side, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, glucocorticoid, auristatin, auromycin, yttrium, bismuth, combrestatin, duocarmycins, dolostatin, cc1065, a cisplatin. auristatin phenylalanine phenylenediamine (AFP), monomethyl auristatin phenylalanine (MMAF), and monomethyl auristatin E (MMAE).

25. The composition of claim 24, wherein the cytotoxic agent is monomethyl auristatin E (MMAE).

26. The composition of claim 1, wherein the active component is linked to the glucan backbone via a payload linker.

27. The composition of claim 26, wherein the payload linker is a cleavable linker or a non-cleavable linker.

28. The composition of claim 27, wherein the cleavable linker is capable of being cleaved by a protease.

29. The composition of claim 28, wherein the protease is a lysosomal protease or an endosomal protease.

30. The composition of claims 27, wherein the cleavable linker is capable of being cleaved by a pH change.

31. The composition of claim 27, wherein the payload linker is a Val-Cit linker.

32. A method of delivering an agent to a macrophage comprising contacting said macrophage with a compound of claim 1.

33. A method of treating cancer in a subject comprising administering to the subject a therapeutically effective amount of a compound of claim 1, wherein the active component is a therapeutic agent.
